(19) United States
(12) Patent Application Publication
(10) Pub. No.: US 2024/0049751 A1
(43) Pub. Date: Feb. 15, 2024

(54) A FEED INGREDIENT COMPRISING PROPIONATE AND A SELECTED BY-PRODUCT OF FERMENTATION

(71) Applicant: WHITE DOGS LABS, INC., New Castle, DE (US)

(72) Inventors: Bryan P. TRACY, Wilmington, DE (US); Aharon M. EYAL, Jerusalem (IL); Shawn William JONES, Bear, DE (US); Biniam MARU, Bear, DE (US); Alon KARPOL, Tel Mond (IL)

(21) Appl. No.: 17/766,243

(22) PCT Filed: Sep. 30, 2020
(86) PCT No.: PCT/IB2020/059129

§ 371 Date: Apr. 3, 2022

Related U.S. Application Data
(60) Provisional application No. 62/909,815, filed on Oct. 3, 2019.

(51) Int. Cl. A23K 10/12; C12P 7/52; A61K 31/19; A23K 20/105

(52) U.S. Cl. CPC A23K 10/12; C12P 7/52; A61K 31/19; A23K 20/105

(57) ABSTRACT

Provided is a feed ingredient comprising propionate and a byproduct of fermentation of a feedstock by propionic acid-producing bacteria, wherein said feedstock comprises a carbon source and wherein said bacteria are of a class selected from the group consisting of Actinobacteria, Bacteroidia, Clostridia, Negativicutes and combinations thereof. Also provided are methods for manufacturing the feed ingredient and uses thereof.

### CROSS-REFERENCE TO RELATED APPLICATION

The present application gains priority from U.S. Provisional Application No. 62/909,815 filed Oct. 3, 2019, which is incorporated by reference as if fully set-forth herein.

### FIELD OF THE INVENTION

The present invention relates to the field of food, and more specifically to a feed ingredient comprising propionate and a byproduct of fermentation of a feedstock by propionic acid-producing bacteria, wherein said feedstock comprises a carbon source and wherein said bacteria are of a class selected from the group consisting of Actinobacteria, Bacteroidia, Clostridia, Negativicutes and combinations thereof.

### BACKGROUND OF THE INVENTION

Animals need a diet that incorporates the four main nutrient groups: protein, carbohydrates, fibers, vitamins, and minerals, in a form which is balanced, digestible and provides all of the necessary nutrients required for an animal's successful development. Animal feeds are preferably selected to support growth and maintain the health of an animal. For livestock farmers, the feed costs generally represent the greatest portion of the overall cost of production, such as up to 70% of the overall production costs, especially for species such as swine or poultry.

There is an ongoing need for improved feed ingredients that can be produced cheaply and efficiently, and which better achieve improvements, such as increased feed intake, improved feed conversion rate, faster daily weight gain, greater ileum surface area and consistent results between sexes.

### SUMMARY OF THE INVENTION

According to an aspect of some embodiments of the present invention, there is provided a feed ingredient comprising propionate and a byproduct of fermentation of a feedstock by propionic acid-producing bacteria, wherein said feedstock comprises a carbon source and wherein said bacteria are of a class selected from the group consisting of Actinobacteria, Bacteroidia, Clostridia, Negativicutes and combinations thereof.

According to an embodiment, said Actinobacteria are of the genus Propionibacterium.

According to an embodiment, said bacteria of the genus Propionibaccterium are of a strain selected from the group consisting of P. acidipropionici, P. shermanii, P. freudenreichii or combinations thereof.

According to an embodiment, said Bacteroidia are of the genus Bacterioides.

According to an embodiment, said Clostridia are of the genus Clostridium.

According to an embodiment, said Negativicutes are of the genus Pactinatus.

According to an embodiment, said byproduct is selected from the group consisting of nitrogen compounds, nucleotides, vitamins, salts, complexes of transition metals, biomass and combinations thereof.

According to an embodiment, said propionate is protected.

According to an embodiment, said propionate forms between 10% by weight and 90% by weight on a dry basis.

According to an embodiment, said byproduct forms between 0.01% by weight and 90% by weight on a dry basis.

According to an embodiment, the feed ingredient comprises less than 20% water.

According to an embodiment, said carbon source is selected from the group consisting of starch, dextrose, maltodextrin, glucose, liquefied corn mash, fructose, xylose, glycerol, sucrose, hemicellulose, cellulose and combinations thereof.

According to an aspect of some embodiments of the present invention, there is provided a method for manufacturing a feed ingredient, the method comprising (i) providing a feedstock comprising a carbon source; (ii) fermenting said feedstock with propionic acid-producing bacteria of a class selected from the group consisting of Actinobacteria, Bacteroidia, Clostridia, Negativicutes and combinations thereof, whereby a biomass propionate and a byproduct is formed.

According to an embodiment, the byproduct is a byproduct of fermentation.

According to an embodiment, said Actinobacteria are of the genus Propionibacterium.

According to an embodiment, said bacteria of the genus Propionibaccterium are of a strain selected from the group consisting of P. acidipropionici, P. shermanii, P. freudenreichii or combinations thereof.

According to an embodiment, said Bacteroidia are of the genus Bacterioides.

According to an embodiment, said Clostridia are of the genus Clostridium.

According to an embodiment, said Negativicutes are of the genus Pactinatus.

According to an embodiment, the method further comprises (iii) separating out said biomass from said fermentation broth to form a clarified fermentation broth, wherein said clarified fermentation broth comprises propionate and said byproduct.

According to an embodiment, said carbon source is selected from the group consisting of starch, dextrose, maltodextrin, glucose, liquefied corn mash, fructose, xylose, glycerol, sucrose, hemicellulose, cellulose and combinations thereof.

According to an embodiment, the method further comprises adding propionate to said clarified fermentation broth.

According to an embodiment, the method further comprises protecting at least a fraction of the propionate in said clarified fermentation broth or in products thereof.

According to an embodiment, the method further comprises at least partially removing water from said biomass-containing fermentation broth and/or from said clarified fermentation broth.

According to an aspect of some embodiments of the present invention, there is provided an animal feed comprising selected nutrients, the feed ingredient as disclosed herein and optionally a biomass.

According to an aspect of some embodiments of the present invention, there is provided a feed ingredient comprising propionate and a by-product of fermentation produced according to the method disclosed herein.

According to an aspect of some embodiments of the present invention, there is provided an animal feed comprising selected nutrients, the feed ingredient as disclosed herein and optionally a biomass.

According to an aspect of some embodiments of the present invention, there is provided a method for treating an animal comprising feeding said animal with the animal feed as disclosed herein.

According to an embodiment, said feed comprises propionate at a concentration between 10 ppm and 20,000 ppm.

According to an embodiment, at least 50% by weight of the propionate in said feed results from said feed ingredient.

According to an embodiment, said animal is selected from the group consisting of an avian, a porcine, a bovine, an equine, an ovine, a piscine, a caprine, a canine, a feline and a lapine.

According to an embodiment, an improved result is obtained compared with that obtained by feeding said selected nutrients without said feed ingredient.

According to an embodiment, said treating comprises increasing feed intake, increasing feed conversion rate, increasing rate of daily weight gain, increasing ileum surface area, and increasing consistency of results between sexes as compared to that obtained by feeding said selected nutrients without said feed ingredient.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIG. 1 is a line graph showing metabolite and cell production and glucose consumed as a function of fermentation time for Acidipropionibacterium acidipropionic batch fermentation with glucose as the feedstock

### DETAILED DESCRIPTION OF THE INVENTION

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

As used herein, the term “feed ingredient” refers to a component, part, constituent or any combination/mixture in an animal food, including a food intended for human consumption.

As used herein, the term “feedstock” refers to a component or components used as a starting material in a fermentation process.

As used herein, the term “animal feed” refers to a food intended for a non-human animal, including but not limited to livestock or poultry.

As used herein, the term “biomass” refers to a cellular mass of microorganisms produced by a fermentation process.

As used herein, the term “protected” with regard to propionate refers to a propionate molecule that is provided in a bound or reversibly reacted form in order to enable the molecule to pass through the upper gastrointestinal tract.

As used herein, the term “treating” includes ameliorating, mitigating, and reducing the instances of a disease or condition, or the symptoms of a disease or condition.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

According to an aspect of some embodiments of the present invention, there is provided a feed ingredient comprising propionate and a byproduct of fermentation of a feedstock by propionic acid-producing bacteria, wherein said feedstock comprises a carbon source and wherein said bacteria are of a class selected from the group consisting of Actinobacteria, Bacteroidia, Clostridia, Negativicutes and combinations thereof.

According to an embodiment, said Actinobacteria are of the genus Propionibacterium.

According to an embodiment, said bacteria of the genus Propionibaccterium are of a strain selected from the group consisting of P. acidipropionici, P. shermanii, P. freudenreichii or combinations thereof.

According to an embodiment, said Bacteroidia are of the genus Bacterioides.

According to an embodiment, said Clostridia are of the genus Clostridium.

According to an embodiment, said Negativicutes are of the genus Pactinatus.

According to an embodiment, said byproduct is selected from the group consisting of nitrogen compounds, nucleotides, vitamins, salts, complexes of transition metals, biomass and combinations thereof.

According to an embodiment, said propionate is protected.

According to an embodiment, said propionate is protected for the transport through the stomach for higher yield of delivery to the intestine. According to an embodiment, said propionate is bound to a glyceride. According to an embodiment, said propionate is encapsulated or coated. According to an embodiment, said propionate is protected by being encapsulated by a protein and/or by being bound to a protein.

According to an embodiment, said propionate forms between 10% by weight and 90% by weight of the feed ingredient on a dry basis. According to an embodiment, said propionate forms at least 15%, at least 20% or at least 30% of the feed ingredient. According to an embodiment, said propionate forms less than 80% of the feed ingredient, less than 70% or less than 60%.

According to an embodiment, said fermentation by-product forms between 0.01% by weight and 90% by weight of the feed ingredient on a dry basis. According to an embodiment, said fermentation byproduct forms at least 0.05%, at least 0.1%, at least 0.2%, at least 1%, at least 2% at least 5% of the feed ingredient.

According to an embodiment, said byproduct is water soluble. According to an embodiment the solubility of said byproduct in water at 20 degrees Celsius is greater than 3% by weight, greater than 5% wt, greater than 7% wt or greater than 9% wt. According to an embodiment, said byproduct is selected from the group consisting of nitrogen compounds, nucleotides, vitamins, salts, complexes of transition metals, biomass and combinations thereof. According to an embodiment, said biomass comprises an organism is selected from the bacteria class of Clostridia. According to an embodiment, said biomass comprises an organism selected from Actinobacteria, Bacteroidia, Clostridia, Negativicutes and combinations thereof.

According to an embodiment, said byproduct is a product of fermenting a feedstock comprising a carbon source. According to an embodiment, said byproduct is a product of fermenting said feedstock with an organism selected from the bacteria class of Clostridia, which organism is further characterized by the ability to produce propionic acid when in native form. According to an embodiment, said organism is selected from Actinobacteria, Bacteroidia, Clostridia, Negativicutes and combinations thereof.

According to an embodiment, said fermented carbon source is selected from the group consisting of starch, dextrose, maltodextrin, glucose, liquefied corn mash, fructose, xylose, glycerol, sucrose, hemicellulose, cellulose and combinations thereof.

According to an embodiment, said feed ingredient comprises less than 20% water, less than 15%, less than 10% or less than 5%.

According to an aspect of some embodiments of the present invention, there is provided a method for manufacturing a feed ingredient, the method comprising (i) providing a feedstock comprising a carbon source; (ii) fermenting said feedstock with propionic acid-producing bacteria of a class selected from the group consisting of Actinobacteria, Bacteroidia, Clostridia, Negativicutes and combinations thereof, whereby a biomass, propionate and a byproduct is formed.

According to an embodiment, the byproduct is a byproduct of fermentation.

According to an embodiment, said Actinobacteria are of the genus Propionibacterium.

According to an embodiment, said bacteria of the genus Propionibaccterium are of a strain selected from the group consisting of P. acidipropionici, P. shermanii, P. freudenreichii or combinations thereof.

According to an embodiment, said Bacteroidia are of the genus Bacterioides.

According to an embodiment, said Clostridia are of the genus Clostridium.

According to an embodiment, said Negativicutes are of the genus Pactinatus.

According to an embodiment, the method further comprises (iii) separating out said biomass from said fermentation broth to form a clarified fermentation broth, wherein said clarified fermentation broth comprises propionate and said byproduct.

According to an embodiment, said carbon source is selected from the group consisting of starch, dextrose, maltodextrin, glucose, liquefied corn mash, fructose, xylose, glycerol, sucrose, hemicellulose, cellulose and combinations thereof.

According to an embodiment, said fermenting is anaerobic. According to an embodiment, said fermenting is conducted at pH greater than 4.8, greater than 5, greater than 5.2, greater than 5.4 or greater than 5.6. According to an embodiment, said fermenting is conducted at a temperature in the range between 25 and 40 degrees Celsius. According to an embodiment, said fermenting is conducted for between 10 hours and 50 hours after inoculation. According to an embodiment, said fermenting is continuous. According to an embodiment, said fermenting is conducted in a fed-batch mode.

According to an embodiment, said method further comprises adding propionate to said clarified filtration broth. According to an embodiment, said method further comprises protecting at least a fraction of the propionate in said clarified fermentation broth or in products thereof.

According to an embodiment, the method further comprises at least partially removing water from said biomass-containing fermentation broth and/or from said clarified fermentation broth.

According to an aspect of some embodiments of the present invention, there is provided an animal feed comprising selected nutrients, the feed ingredient as disclosed herein and optionally a biomass.

According to an aspect of some embodiments of the present invention, there is provided a feed ingredient comprising propionate and a by-product of fermentation produced according to the method disclosed herein.

According to an aspect of some embodiments of the present invention, there is provided an animal feed comprising selected nutrients, the feed ingredient as disclosed herein and optionally a biomass.

According to an embodiment, said biomass comprises bacteria of a class selected from the group consisting of Actinobacteria, Bacteroidia, Clostridia, Negativicutes and combinations thereof.

According to an embodiment, said nutrients comprise macronutrients comprising energy sources (such as carbon sources, fats and proteins) and nitrogen sources (such as proteins and amino acids) and/or micronutrients (such as vitamins, minerals and trace elements).

According to an embodiment, said feed comprises propionate at a concentration between 10 ppm and 20,000 ppm. According to an embodiment, said feed comprises at least 50 ppm propionate, at least 100 ppm, at least 300 ppm, at least 500 ppm propionate, at least 700 ppm, or at least 1000 ppm. According to an embodiment, at least 50% by weight of the propionate in said feed results from said feed ingredient, at least 60%, at least 70% or at least 80%.

According to an embodiment, provided is a method for treating an animal comprising feeding said animal with the animal feed as disclosed herein. According to an embodiment, there is provided an animal feed as disclosed herein for use in treating an animal. According to an embodiment, said animal is selected from the group consisting of avian, porcine, bovine, equine, ovine, piscine, caprine, canine, feline and lapine animals. According to an embodiment, said treating comprises increasing feed intake, increasing feed conversion rate, increasing rate of daily weight gain, increasing ileum surface area, and increasing consistency of results between sexes as compared to that obtained by feeding said selected nutrients without said feed ingredient.

According to an embodiment, the animal feed comprising the feed ingredient as disclosed herein is the sole animal feed provided to the animal.

According to an embodiment, said improved result comprises a characteristic selected from the group consisting of increased feed intake, improved feed conversion rate, faster daily weight gain, greater ileum surface area, consistent results between sexes and combinations thereof. According to an embodiment, said improved result comprises at least two of said characteristic, at least three or at least four.

According to an embodiment, said method is characterized by an improved result compared with feeding same selected nutrients without said feed ingredient. According to an embodiment, said improved result comprises increased feed intake. According to an embodiment, feed intake increases by at least 0.5%, at least 1%, at least 2%, at least 3%, or at least 4%. According to an embodiment, said improved result comprises improved feed conversion rate (FCR). According to an embodiment, FCR improves by at least 0.2%, at least 1%, at least 2.5% or at least 10%. According to an embodiment, said improved result comprises faster daily weight gain. According to an embodiment, FCR improves by at least 0.2%, at least 1%, at least 2.5% or at least 10%.

According to an embodiment, provided is a method for manufacturing a feed ingredient, comprising (i) providing a feedstock comprising a carbon-source; (ii) fermenting said feedstock with propionic acid-producing bacteria of a class selected from the group consisting of Actinobacteria, Bacteroidia, Clostridia, Negativicutes and combinations thereof, whereby a fermentation broth comprising a biomass, propionate and a by-product is formed; (iii) separating out said biomass from said fermentation broth to form a said feed ingredient comprising propionate and said by-product. According to an embodiment, said separating comprises at least one of filtering and centrifugation.

According to an embodiment, said carbon source is selected from the group consisting of starch, dextrose, maltodextrin, glucose, liquefied corn mash, fructose, xylose, glycerol, sucrose, hemicellulose, cellulose and combinations thereof.

According to an embodiment, said fermenting is conducted at pH greater than 4.8, greater than 5, greater than 5.2, greater than 5.4 or greater than 5.6. According to an embodiment, said fermenting is conducted at a temperature in the range between 25 and 40 degrees Celsius. According to an embodiment, said fermenting is conducted for between 10 hours and 50 hours after inoculation. According to an embodiment, said fermenting is continuous. According to an embodiment, said fermenting is conducted in a fed-batch mode.

According to an embodiment, said method further comprises adding propionate to said clarified filtration broth. According to an embodiment, said method further comprises protecting at least a fraction of the propionate in said clarified filtration broth or in products thereof. According to an embodiment, said method further comprises water removal from said biomass and/or from said clarified filtration broth, at least partially. According to an embodiment, said water removal comprises water evaporation.

According to an aspect of some embodiments of the present invention, there is provided an animal feed comprising a feed ingredient manufactured according to the method as disclosed herein or a product thereof, selected nutrients, and optionally biomass.

According to an aspect of some embodiments of the present invention, there is provided a method for treating an animal comprising feeding said animal with the feed as disclosed herein

According to an embodiment, said biomass comprises an organism is selected from the group consisting of Actinobacteria, Bacteroidia, Clostridia, Negativicutes and combinations thereof,

According to an embodiment, said nutrients comprise energy sources and protein sources. According to an embodiment, said feed comprises propionate at a concentration between 10 ppm and 20,000 ppm. According to an embodiment, said feed comprises at least 50 ppm propionate, at least 100 ppm, at least 300 ppm, at least 500 ppm propionate, at least 700 ppm, or at least 1000 ppm. According to an embodiment, at least 50% by weight of the propionate in said feed results from said feed ingredient, at least 60%, at least 70% or at least 80%.

According to an embodiment, provided is a method for treating an animal comprising feeding said animal with the feed as disclosed herein. According to an embodiment, said animal is selected from the group consisting of avian, porcine, bovine, equine, ovine, piscine, caprine, canine, feline and lapine animals.

According to an embodiment, said improved result comprises at least one characteristic selected from the group consisting of increased feed intake, improved feed conversion rate, faster daily weight gain, greater ileum surface area, and consistent results between sexes. According to an embodiment, said improved result comprises at least two of said characteristic, at least three or at least four.

According to an embodiment, said method is characterized by an improved result compared with feeding same selected nutrients without said feed ingredient. According to an embodiment, said improved result comprises increased feed intake. According to an embodiment, feed intake increases by at least 0.5%, at least 1%, at least 2%, at least 3%, or at least 4%. According to an embodiment, said improved result comprises improved feed conversion rate (FCR). According to an embodiment, FCR improves by at least 0.1%, at least 0.2%, at least 0.3%, or at least 0.4%. According to an embodiment, said improved result comprises faster daily weight gain. According to an embodiment, daily weight gain increases by at least 0.1%, at least 0.2%, at least 0.3%, or at least 0.4%.

### EXAMPLES

### Example 1: Feedstock Fermentation

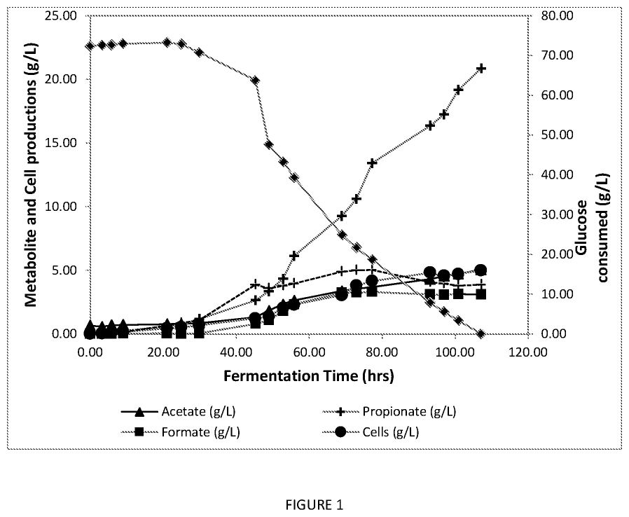

Glucose was fermented in a batch fermentation with Propionibacterium acidipropionici (also known as Acidipropionibacterium acidipropionici). The fermentation was kept anaerobic by continuous nitrogen sparging. Temperature was maintained at about 37° C. and pH was controlled at a minimum of 6.5 with NH 4 OH. The fermentation was run until the glucose was fully consumed, Results of glucose consumption, cell production and metabolites production during 107 hours of fermentation are shown in FIG. 1. Propionate concentration and cell concentration reached about 20.8 g/L and 5 g/L, respectively.

### Example 2: Manufacture of the Feed Ingredient

Fermentation was conducted similarly to Example 1. Biomass was filtered out. The filtrate was concentrated up by water evaporation to reach total solids concentration of 11.16% by weight to form the feed ingredient, having a density of 1.086 Kg/L. The formed feed ingredient was analyzed. The results are summarized in Table 1.

TABLE 1
Exemplary composition of propionate feed ingredient

| Component | Value | Units |
|---|---|---|
| Total solids | 53.7 | wt % |
| Density | 1.078 | kg/L |
| Ammonium Propionate | 281.25 | g/kg |
| Ammonium Acetate | 81.17 | g/kg |
| Ammonium Lactate | 47.36 | g/kg |
| Ammonium Formate | 45.31 | g/kg |
| Crude Fat | 3.22 | g/kg |
| Ash | 28.89 | g/kg |
| Calcium | 0.27 | g/kg |
| Phosphorous | 0.32 | g/kg |
| Magnesium | 0.16 | g/kg |
| Potassium | 5.26 | g/kg |
| Sodium | 5.91 | g/kg |
| Iron | 31.68 | ppm |
| Zinc | 0.04 | ppm |
| Copper | <1 | ppm |
| Manganese | 53.57 | ppm |
| Molybdenum | 0.62 | ppm |
| Sulfur | 2700.00 | ppm |

1. A feed ingredient comprising propionate and a byproduct of fermentation of a feedstock by propionic acid-producing bacteria, wherein said feedstock comprises a carbon source and wherein said bacteria are of a class selected from the group consisting of Actinobacteria, Bacteroidia, Clostridia, Negativicutes and combinations thereof.

2. The feed ingredient of claim 1, wherein said Actinobacteria are of the genus Propionibacterium, optionally of a strain selected from the group consisting of P. acidipropionici, P. shermanii, P. freudenreichii or combinations thereof.

3. (canceled)

4. The feed ingredient of claim 1, wherein said Bacteroidia are of the genus Bacterioides; said Clostridia are of the genus Clostridium; and said Negativicutes are of the genus Pactinatus.

5. (canceled)

6. (canceled)

7. The feed ingredient of claim 1, wherein said byproduct is selected from the group consisting of nitrogen compounds, nucleotides, vitamins, salts, complexes of transition metals, biomass and combinations thereof.

8. (canceled)

9. The feed ingredient of claim 1, wherein said propionate forms between to % by weight and 90% by weight on a dry basis of the total weight of the feed ingredient.

10. The feed ingredient of claim 1, wherein said byproduct forms between 0.01% by weight and 90% by weight on a dry basis of the total weight of the feed ingredient.

11. The feed ingredient of claim 1, comprising less than 20% water.

12. The feed ingredient of claim 1, wherein said carbon source is selected from the group consisting of starch, dextrose, maltodextrin, glucose, liquefied corn mash, fructose, xylose, glycerol, sucrose, hemicellulose, cellulose and combinations thereof.

13. A method for manufacturing a feed ingredient, the method comprising
(i) providing a feedstock comprising a carbon source;
(ii) fermenting said feedstock with propionic acid-producing bacteria of a class selected from the group consisting of Actinobacteria, Bacteroidia, Clostridia, Negativicutes and combinations thereof, whereby a fermentation broth comprising a biomass, propionate and a byproduct is formed, wherein said byproduct is optionally a byproduct of fermentation.

14. (canceled)

15. The method of claim 13, wherein said Actinobacteria are of the genus Propionibacterium, optionally of a strain selected from the group consisting of P. acidipropionici, P. shermanii, P. freudenreichii or combinations thereof.

16. (canceled)

17. The method of claim 13, wherein said Bacteroidia are of the genus Bacterioides; said Clostridia are of the genus Clostridium; and said Negativicutes are of the genus Pactinatus.

18. (canceled)

19. (canceled)

20. The method of claim 13, further comprising (iii) separating out said biomass from said fermentation broth to form a clarified fermentation broth, wherein said clarified fermentation broth comprises said propionate and said byproduct.

21. The method of claim 13, wherein said carbon source is selected from the group consisting of starch, dextrose, maltodextrin, glucose, liquefied corn mash, fructose, xylose, glycerol, sucrose, hemicellulose, cellulose and combinations thereof.

22. The method of claim 13, further comprising adding propionate to said clarified fermentation broth, and optionally further comprising protecting at least a fraction of the propionate in said clarified fermentation broth or in products thereof.

23. (canceled)

24. The method of claim 22, further comprising at least partially removing water from said biomass-containing fermentation broth and/or from said clarified fermentation broth.

25. (canceled)

26. An animal feed comprising selected nutrients, said feed ingredient of claim 1 and optionally a biomass.

27. A method for treating an animal comprising feeding said animal with the feed of claim 26, wherein the animal is optionally selected from the group consisting of an avian, a porcine, a bovine, an equine, an ovine, a piscine, a caprine, a canine, a feline and a lapine.

28. The method of claim 27, wherein said feed comprises propionate at a concentration between 10 ppm and 20,000 ppm.

29. The method of claim 27, wherein at least 50% by weight of the propionate in said feed results from said feed ingredient.

30. (canceled)

31. The method of claim 27, wherein said treating comprises increasing feed intake, increasing feed conversion rate, increasing rate of daily weight gain, increasing ileum surface area, and increasing consistency of results between sexes as compared to that obtained by treating said animal with said selected nutrients in the absence of said feed ingredient.
